(19) United States
(12) Patent Application Publication
(10) Pub. No.: US 2016/0331266 A1
(43) Pub. Date: Nov. 17, 2016

(54) BIOLOGICAL IMPEDANCE MEASUREMENT PROBE, MEASUREMENT SYSTEM AND METHOD BASED ON SPECTRAL CHARACTERISTIC

(71) Applicant: SEALAND TECHNOLOGY (CHENGDU) LIMITED, Chengdu, Sichuan (CN)

(72) Inventors: Fei Xiang, Chengdu (CN); Yigang Wang, Chengdu (CN); Tao Dai, Chengdu (CN); Xianhong Xu, Chengdu (CN); Pu Yang, Chengdu (CN); Song Gao, Chengdu (CN); Lining Bu, Chengdu (CN); Yi Lin, Chengdu (CN)

(21) Appl. No.: 15/111,092

(22) PCT Filed: Mar. 18, 2015
(86) PCT No.: PCT/CN2015/074484

§ 371 Date: Jul. 12, 2016

(30) Foreign Application Priority Data
Apr. 3, 2014 (CN) 201410133222.0

(51) Int. Cl. A61B 5/053; A61B 5/00

(52) U.S. Cl. CPC A61B 5/053; A61B 5/7225; A61B 5/7264; A61B 2562/0209

(57) ABSTRACT

A measurement probe includes a substrate and six electrodes embedded in the substrate, including a first electrode, a second electrode, a third electrode, a fourth electrode, a fifth electrode and a sixth electrode, the first electrode is arranged opposite to the fourth electrode, and/or the second electrode is arranged opposite to the fifth electrode, and/or and the third electrode is arrange opposite to the sixth electrode. Excitations are applied between any opposite electrodes, the electrodes are circumferentially distributed on the substrate, and at least two pairs of electrodes are in axial symmetry. The probe can carry out stable signal collection and has simple structure. The measurement system and the measurement method can reduce the influence of the contact impedance between the probe and the measured tissues and also improve the accuracy of judging whether the probe is located at the juncture of human body tissues or animal body tissues.

### FIELD OF THE INVENTION

The present invention relates to technical field of biological impedance measurement, and more particularly to a biological impedance measurement probe based on spectral characteristic, a measurement system and a method accordingly.

### BACKGROUND OF THE INVENTION

Impedance frequency characteristic of biological tissues is also called as impedance spectroscopy characteristic, which means that values of the resistive and capacitive components of the biological tissues markedly vary with the frequency of the electrical signal. Since the bioelectrical impedance is in close correlation to the measurement frequency, and the impedance variation characteristic is in close correlation to its cellular morphology, arrangement of cells, content of stromal cells and electrolyte concentration, in the audio frequency range, thus it's valuable to obtain the electrical impedance characteristics of the tissues or organs in the frequency range, so as to get the tissue state, assess organ function, and identify diseased tissue; and further it's potential to be developed in health status assessment, early diagnosis, and drug efficacy monitoring and critical disease monitoring, etc.

Current technology developed a method to measure tissue impedance for patients or monitor pathology or physiological conditions by applying a safe electrical current to biological tissues through electrodes, and the biological tissues can be breast tissues, or cervix tissues, and the like. For example, when a probe with electrodes is used to perform cervical cancer screening, two main normal tissues of squamous epithelium tissues and columnar structure will be identified in the impedance spectrum. Since the impedance spectrum of the precancerous tissue is between the impedance spectrum of the normal squamous epithelium tissues and normal columnar structure, when the probe is placed on the uterine cavity that is located a juncture of the two types of issues, the impedance measured by the probe looks like matching with the precancerous tissue. Therefore, the position of the probe is important to the measurement, if the probe is placed on an inappropriate position, a wrong diagnosis result will be generated. Thus it's desired to develop a measurement probe that has stable collection signals and multiple collection manners to facilitate the subsequent analysis.

### SUMMARY OF THE INVENTION

To solve the technical problems mentioned above, the objective of the present invention is to provide a biological impedance measurement probe based on spectral characteristic that has simple structure and stable signal collection, the probe includes a substrate and at least six electrodes embedded in the substrate, the six electrodes includes a first electrode, a second electrode, a third electrode, a fourth electrode, a fifth electrode and a sixth electrode, the first electrode is arranged opposite to the fourth electrode, and/or the second electrode is arranged opposite to the fifth electrode, and/or and the third electrode is arrange opposite to the sixth electrode. Excitations are applied between any opposite electrodes, the electrodes are circumferentially distributed on the substrate, and at least two pairs of electrodes are in axial symmetry.

Preferably, surfaces of the six electrodes are flush with a surface of the substrate, and spaces between two adjacent electrodes are identical.

Preferably, a central angle between two electrodes that are opposite to each other is 180°.

To solve the technical problems mentioned above, the objective of the present invention is to provide a biological impedance measurement system based on spectral characteristic that has simple structure and stable signal collection, the system includes:

a probe, comprising a substrate and at least six electrodes embedded in the substrate, the six electrodes including a first electrode, a second electrode, a third electrode, a fourth electrode, a fifth electrode and a sixth electrode;

an excitation source, adapted for applying N excitations with different frequencies to the first electrode and the fourth electrode that are opposite to each other, the second electrode and the fifth electrode that are opposite to each other, and the third electrode and the sixth electrode that are opposite to each other, and the frequencies being expressed by fᵢ, i=1, 2 3 . . . N;

a signal collection circuit, adapted for measuring a first electrical parameter D₁ᵢ between the second electrode and the third electrode, a second electrical parameter D₂ᵢ between the fifth electrode and the sixth electrode, a third electrical parameter D₃ᵢ between the third electrode and the fourth electrode, a fourth electrical parameter D₄ᵢ between the first electrode and the sixth electrode, a fifth electrical parameter D₅ᵢ between the fourth electrode and the fifth electrode, and a sixth electrical parameter D₆ᵢ between the first electrode and the second electrode;

a multiple-selection switch system, adapted for controlling the connection and disconnection among the six electrodes, the excitation source and the signal collection circuit; and

a spectrum analyzer, adapted for recording and storing three pairs of the electrical parameters D₁ᵢ and D₂ᵢ, D₃ᵢ and D₄ᵢ, D₅ᵢ and D₆ᵢ, and performing logic judgment and statistical analysis to the three pairs of the electrical parameter.

Preferably, surfaces of the six electrodes are flush with a surface of the substrate, and spaces between two adjacent electrodes are identical.

Preferably, a central angle between two electrodes that are opposite to each other is 180°.

To solve the technical problems mentioned above, the objective of the present invention is to provide a biological impedance measurement method based on spectral characteristic that has simple structure and stable signal collection, the method includes:

step 1, disposing a probe on biological tissues, with the probe contacting with the biological tissues;

step 2, selecting N frequencies in a testing frequency range of fₘ˜fₙ, and selecting an excitation source with a frequency fᵢ ∈[fₘ, fₙ], and =1, 2, 3 . . . N, fₘ<fₙ

step 3, controlling and respectively applying excitations with N frequencies to the first electrode and the fourth electrode, the second electrode and the fifth electrode, and the third electrode and the sixth electrode, by means of the multiple-selection switch system; collecting a first electrical parameter D₁ᵢ between the second and the third electrodes and a second electrical parameter D₂ᵢ between the fifth and the sixth electrodes when the excitation is applied to the first electrode and the fourth electrode, a third electrical parameter D₃ᵢ between the third and the fourth electrodes and a fourth electrical parameter D₄ᵢ between the first and the sixth electrodes when the excitation is applied to the second and the fifth electrodes, and a fifth electrical parameter D₅ᵢ between the fourth and the fifth electrodes and a sixth electrical parameter D₆ᵢ between the first and the second electrodes when the excitation is applied to the third and the sixth electrodes, by the signal collection circuit;

step 4, performing curve fitting to N first electrical parameters D₁ᵢ and N second electrical parameters D₂ᵢ, N third electrical parameters D₃ᵢ and N fourth electrical parameters D₄ᵢ, N fifth electrical parameters D₅ᵢ and N sixth electrical parameters D₆ᵢ to obtain three pairs of biological impedance spectrum curves; and

step 5, analyzing the three pairs of biological impedance spectrum curves by means of weighting methods to determine whether the biological tissues are different or not.

Preferably, the method further includes obtaining N first electrical parameters D₁ᵢ between the second and the third electrodes and performing statistical analysis to the N first electrical parameters D₁ᵢ to obtain a curve a, and obtaining N second electrical parameters D₂ᵢ between the fifth and the sixth electrodes and performing statistical analysis to the N second electrical parameters D₂ᵢ to obtain a curve b, when excitations with N different frequencies fᵢ are applied to the first and the fourth electrodes, wherein the curves a and b constitute a first pair of biological impedance spectrum curve.

Preferably, the method further includes obtaining N third electrical parameters D₃ᵢ between the third and the fourth electrodes and performing statistical analysis to the N third electrical parameters D₃ᵢ to obtain a curve c, and obtaining N fourth electrical parameters D₄ᵢ between the first and the sixth electrodes and performing statistical analysis to the N fourth electrical parameters D₄ᵢ to obtain a curve d, when excitations with N different frequencies fᵢ are applied to the second and the fifth electrodes, wherein the curves c and d constitute a second pair of biological impedance spectrum curve.

Preferably, the method further includes obtaining N fifth electrical parameters D₅ᵢ between the fourth and the fifth electrodes and performing statistical analysis to the N fifth electrical parameters D₅ᵢ to obtain a curve e, and obtaining N sixth electrical parameters D₆ᵢ between the first and the second electrodes and performing statistical analysis to the N sixth electrical parameters D₆ᵢ to obtain a curve f, when excitations with N different frequencies fᵢ are applied to the third and the sixth electrodes, wherein the curves e and f constitute a third pair of biological impedance spectrum curve

Advantages of the present invention includes:

1. The surfaces of the electrodes of the probe according to the present invention are flush with the surface of the substrate, thus data collected by every electrode are consistent, and therefore the signal collection is accurate and stable.

2. The probe of the present invention has at least six electrodes which are circumferentially distributed on the substrate, with an identical space, thus the collection modes are multiple which is beneficial to perform comparison analysis among the collection modes.

3. The system and method of the present invention can prevent measurement error caused by different organism tissues, the probe can judge whether the measured organism are the same organism or not, without moving, thus the signal collection is stable and simply, and the measurement result is accurate.

4. The system and method of the present invention can select multiple frequencies in a certain range, in the form of logarithm, three different measurement modes can be applied, and six groups of impedance data can be measured accordingly, thus it's beneficial to distinguish the different biological tissues and analyze the data.

5. The system and method of the present invention can reduce the contacting impedance between the probe and the juncture of the different biological tissues, and biological impedance spectrum curves can be obtained by means of statistical analysis, and different states of the probe and the juncture of the different biological tissues can be determined by means of weighting method, thus the using scope is wide and the operation difficulty is reduced.

### BRIEF DESCRIPTION OF THE DRAWINGS

FIG. 1 is a structure view of a probe according to one embodiment of the present invention;

FIG. 2 is a schematic view showing the probe and the juncture of different tissues are under Q1 state;

FIG. 3 is a schematic view showing the probe and the juncture of different tissues are under Q2 state;

FIG. 4 is a schematic view showing the probe and the juncture of different tissues are under Q3 state;

FIG. 5 is structure view of a measurement system according to one embodiment of the present invention;

FIG. 6 is a flowchart of a measurement method according to one embodiment of the present invention;

FIG. 7 is a schematic diagram showing three pairs of biological impedance spectrum curves for different biological tissues by measuring the state of FIG. 2 according to the measurement method of the present invention;

FIG. 8 is a schematic diagram showing three pairs of biological impedance spectrum curves for different biological tissues by measuring the state of FIG. 3 according to the measurement method of the present invention;

FIG. 9 is a schematic diagram showing three pairs of biological impedance spectrum curves for different biological tissues by measuring the state of FIG. 4 according to the measurement method of the present invention;

FIG. 10 is a schematic diagram showing three pairs of biological impedance spectrum curves for different biological tissues obtained by the measurement method according to one embodiment of the present invention;

FIG. 11 is a schematic diagram showing three pairs of biological impedance spectrum curves for the congeneric biological tissues obtained by the measurement method according to one embodiment of the present invention;

FIG. 12 is a schematic diagram showing three pairs of biological impedance spectrum curves for the congeneric biological tissues obtained by the measurement method according to another embodiment of the present invention; and

FIG. 13 is a structure view of a probe according to another embodiment of the present invention.

The labels includes:

10—probe, 11—substrate, 12—first electrode, 13—second electrode, 14—third electrode, 15—fourth electrode, 16—fifth electrode, 17—sixth electrode, 20—multiple-selection switch system, 30—excitation source, 40—signal collection circuit, 50—spectrum analyzer, Q1—juncture of different tissues is located at or close to the center of the probe, Q2—juncture of different tissues is far from the center or the edge of the probe, Q3—juncture of different tissues is located at or close to the edge of the probe, a, b, c, d, e, f—biological impedance spectrum curves.

### DETAILED DESCRIPTION OF ILLUSTRATED EMBODIMENTS

Following is the preferred embodiments of the present invention, which will not limit the scope of the present invention however.

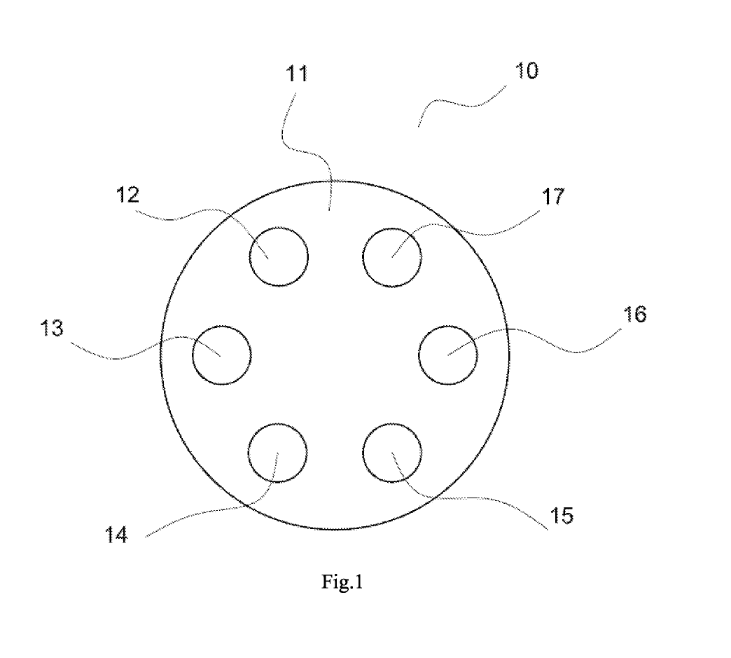

As shown in FIG. 1, a biological impedance measurement probe 10 based on spectral characteristic includes a substrate 11 and at least six electrodes embedded in the substrate 11. The six electrodes includes a first electrode 12, a second electrode 13, a third electrode 14, a fourth electrode 15, a fifth electrode 16 and a sixth electrode 17, the first electrode 12 is arranged opposite to the fourth electrode 15, the second electrode 13 is arranged opposite to the fifth electrode 16, and the third electrode 14 is arrange opposite to the sixth electrode 17. In this disclosure, the opposite arrangements of the electrodes mean that, two electrodes are distributed at two sides of a connecting line between the first electrode 12 and the fourth electrode 15, two electrodes are distributed at two sides of a connecting line between the second electrode 13 and the fifth electrode 16, and two electrodes are distributed at two sides of a connecting line between the third electrode 14 and the sixth electrode 17.

Specifically, an excitation is applied to every two electrodes that are opposite to each other.

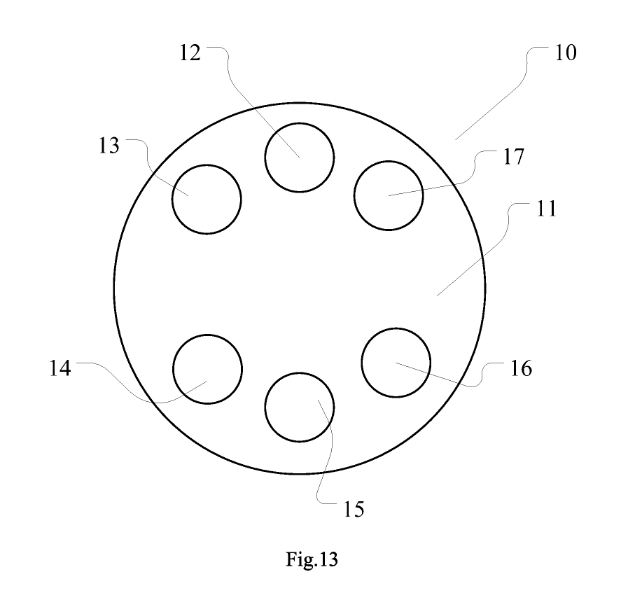

In an embodiment, the electrodes are circumferentially distributed on the substrate 11. In another embodiment, the electrodes are circumferentially distributed on the substrate 11 and have at least two pairs of electrodes that are in axial symmetry, referring to FIG. 13.

For ensuring a full contact between the probe 11 and the measured tissues, and ensuring accuracy of the data collection, the surfaces of the electrodes are flush with the surface of the substrate 11, and the spaces between two adjacent electrodes are the same, namely the electrodes are uniformly distributed on the substrate 11 circumferentially. In this embodiment, the central angle between the two electrodes that are opposite to each other is 180°, in other words, the center of the circle is located in the connecting line between the two opposite electrodes, in such a way, the data collection is convenient.

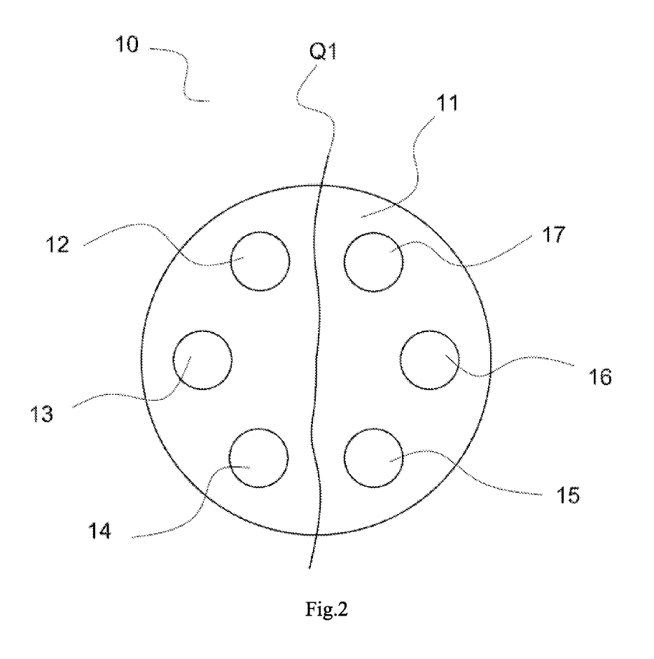

As shown in FIGS. 2-4, the contacting states between the probe 10 and the juncture of different biological tissues generally includes three states Q1, Q2 and Q3. In the present invention, the probe 10 can apply excitations between the first electrode 12 and the fourth electrode 15, between the second electrode 13 and the fifth electrode 16, or between the third electrode 14 and the sixth electrode 17, so that three groups of data can be collected by the probe 10 under the action of the excitation source with a certain frequency. If different frequencies are applied, then three different groups of data will be collected, thus it's easy to analyze and obtain biological impedance spectrum curves for the measured tissues, whereby the accuracy of determining the juncture of different biological tissues for human body or animal body is improved, and the diagnosis of pathological biological tissue is facilitated accordingly.

Comparing with the probe of the present invention and the conventional probe with four electrodes, the collection speeds are similar, nevertheless, the collection manners of the present invention are multiple, the data collected from the measured tissues are comprehensive, which may greatly reduce the influence of disturbances resulted from the actual clinical environmental conditions and operating procedures, etc., and the juncture of the measured tissues can be measured in real time.

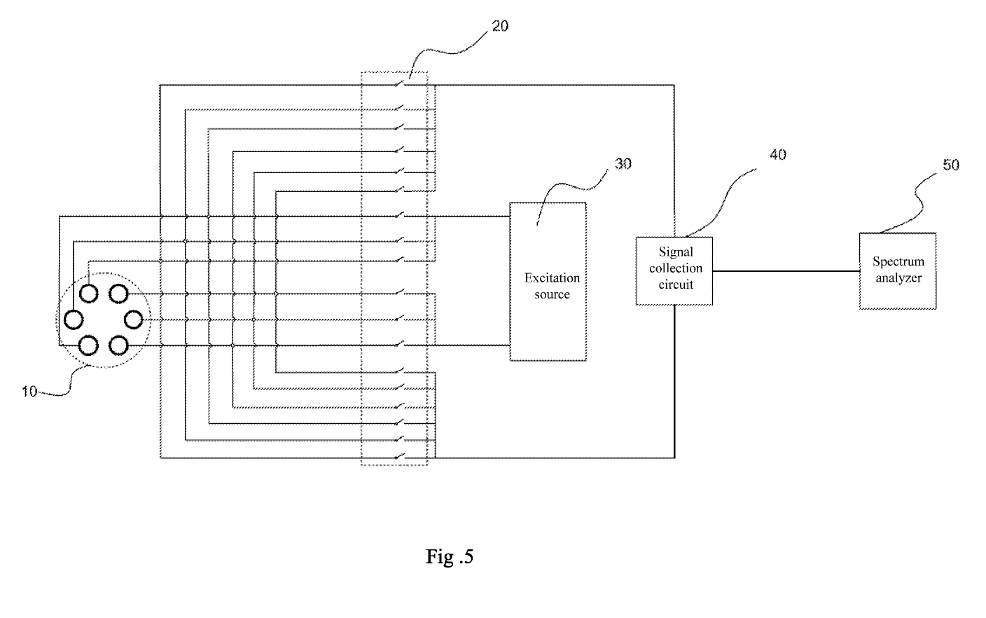

By combining the FIGS. 1 and 5, a measurement system based on spectral characteristic of the present invention includes:

the probe 10;

an excitation source 30, adapted for applying N excitations with different frequencies fᵢ to the first electrode 12 and the fourth electrode 15 that are opposite to each other, the second electrode 13 and the fifth electrode 16 that are opposite to each other, and the third electrode 14 and the sixth electrode 17 that are opposite to each other, therein i=1, 2, 3 . . . N;

a signal collection circuit 40, adapted for measuring a first electrical parameter D₁ᵢ between the second electrode and the third electrode, a second electrical parameter D₂ᵢ between the fifth electrode and the sixth electrode, a third electrical parameter D₃ᵢ between the third electrode and the fourth electrode, a fourth electrical parameter D₄ᵢ between the first electrode and the sixth electrode, a fifth electrical parameter D₅ᵢ between the fourth electrode and the fifth electrode, and a sixth electrical parameter D₆ᵢ between the first electrode and the second electrode;

a multiple-selection switch system 20, adapted for controlling the connection and disconnection among the six electrodes, the excitation source 30 and the signal collection circuit 40; and

a spectrum analyzer 50, adapted for recording and storing the three groups of electrical parameters D₁ᵢ and D₂ᵢ, D₃ᵢ and D₄ᵢ, D₅ᵢ and D₆ᵢ, and performing logic judgment and statistical analysis to them to form biological impedance spectrum curves. Specifically, the electrical parameters D₁ᵢ and D₂ᵢ, D₃ᵢ and D₄ᵢ, D₅ᵢ and D₆ᵢ are shown as the following table 1.

For ensuring a full contact between the probe and the measured tissues, and ensuring the accuracy of the data collection, the surfaces of the electrodes are flush with the surface of the substrate 11, and the spaces between two adjacent electrodes are the same. In the present embodiment, the central angle between two opposite electrodes is 180° , so as to collect the data preferably.

The measurement system of the present invention can obtain three groups of electrical parameters by applying excitations between the opposite electrodes based on a probe with six electrodes, and also can obtain multiple three-group electrical parameters by applying excitations with different frequencies, thus it's accurate to determine the juncture of different biological tissues for human body or animal body, and it's facilitated to carry out the diagnosis of pathological biological tissues. By comparing the multiple groups of collection data, the influences of the contact impedance resulted from the surface smoothness and different pH of the tissues can be avoided.

Therefore, the collection manners of the present invention are multiple, the data collected from the measured tissues are comprehensive, which may greatly reduce the influence of disturbances resulted from the actual clinical environmental conditions and operating procedures, etc., and the juncture of the measured tissues can be measured in real time.

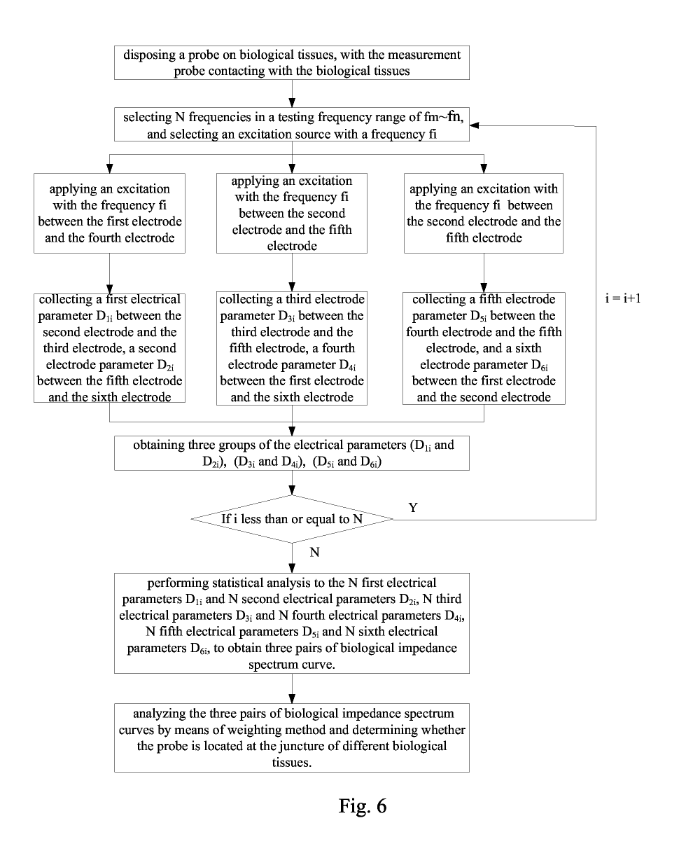

As shown in FIG. 6, a measurement method by means of the measurement system based on spectral characteristic includes the following steps.

S1, disposing a probe 10 on biological tissues, with the measurement probe contacting with the biological tissues;

S2, selecting N frequencies in a testing frequency range of fₘ˜fₙ, and selecting an excitation source 30 with a frequency fᵢ ∈[fₘ, fₙ[, and i=1, 2, 3 . . . N, fₘ<fₙ;

S3, when i =1, fᵢ=fᵢ, applying an excitation with the frequency fᵢ between the first electrode 12 and the fourth electrode 15, between the second electrode 13 and the fifth electrode 16, and between the third electrode 14 and the sixth electrode 17, by the multiple-selection switch system 20;

S4, collecting a first electrical parameter D₁ᵢ between the second electrode 13 and the third electrode 14, a second electrode parameter D₂ᵢ between the fifth electrode 16 and the sixth electrode 17, a third electrode parameter D₃ᵢ between the third electrode 14 and the fifth electrode 15, a fourth electrode parameter D₄ᵢ between the first electrode 12 and the sixth electrode 17, a fifth electrode parameter D₅ᵢ between the fourth electrode 15 and the fifth electrode 16, and a sixth electrode parameter D₆ᵢ between the first electrode 12 and the second electrode 13, by the signal collection circuit;

S5, storing and recording three groups of the electrical parameters D₁ᵢ and D₂ᵢ, D₃ᵢ and D₄ᵢ, D₅ᵢ and D₆ᵢ;

S6, when i≦N, i=i+1, repeating the step S3 to step S5 to obtain the collection data shown in table 1.

The following table 1 shows the collection data for three groups of electrical parameters (D₁ᵢ and D₂ᵢ, D₃ᵢ and D₄ᵢ, D₅ᵢ and D₆) measured by three collection modes, by applying an excitation source with N different frequencies fᵢ.

TABLE 1

| | | | | | | | |
|---|---|---|---|---|---|---|---|
| i | fᵢ | D₁ᵢ | D₂ᵢ | D₃ᵢ | D₄ᵢ | D₅ᵢ | D₆ᵢ |
| 1 | f₁ | D₁₁ | D₂₁ | D₃₁ | D₄₁ | D₅₁ | D₆₁ |
| 2 | f₂ | D₁₂ | D₂₂ | D₃₂ | D₄₂ | D₅₂ | D₆₂ |
| . | . | . | . | . | . | . | . |
| . | . | . | . | . | . | . | . |
| . | . | . | . | . | . | . | . |
| N | f_{N} | D_{1N} | D_{2N} | D_{3N} | D_{4N} | D_{5N} | D_{6N} |

S7, performing statistical analysis to the N first electrical parameters D₁ᵢ and N second electrical parameters D₂ᵢ, N third electrical parameters D₃ᵢ and N fourth electrical parameters D₄ᵢ, N fifth electrical parameters D₅ᵢ and N sixth electrical parameters D₆ᵢ, to obtain three pairs of biological impedance spectrum curve.

In the condition of excitations with N different frequencies fᵢ are applied, the three modes of data collections at three pairs of electrodes are as following.

When the excitation is applied between the first electrode 12 and the fourth electrode 15, N first electrical parameters D₁ᵢ between the second electrode 13 and the third electrode 14 are measured, and a curve a is obtained by performing statistical analysis to the electrical parameters D₁ᵢ; and N second electrical parameters D₂ᵢ between the fifth electrode 16 and the sixth electrode 17 are measured, and a curve b is obtained by performing statistical analysis to the electrical parameters D₂ᵢ; in such a way, the curves a and b form the first pair of biological impedance spectrum curve. Specifically, taking the curve a as a sample, the N first electrical parameters D₁ᵢ and the corresponding frequencies fᵢ are potted on the coordinate plane whose abscissa indicates the frequency and ordinate indicates the electrical parameters for example, so that the curve a can be fitted. In other embodiments, numerical analysis means can also be used to obtain the fitted curve according to the acquired electrical parameters.

When the excitation is applied between the second electrode 13 and the fifth electrode 16, N third electrical parameters D₃ᵢ between the third electrode 14 and the fourth electrode 15 are measured, and a curve c is obtained by performing statistical analysis to the electrical parameters D₃ᵢ; and N fourth electrical parameters D₄ᵢ between the first electrode 12 and the sixth electrode 17 are measured, and a curve d is obtained by performing statistical analysis to the electrical parameters D₄ᵢ; in such a way, the curves c and d form the second pair of biological impedance spectrum curve.

When the excitation is applied between the third electrode 14 and the sixth electrode 17, N fifth electrical parameters D₅ᵢ between the fourth electrode 15 and the fifth electrode 16 are measured, and a curve e is obtained by performing statistical analysis to the electrical parameters D₅ᵢ; and N sixth electrical parameters D₆ᵢ between the first electrode 12 and the second electrode 13 are measured, and a curve f is obtained by performing statistical analysis to the electrical parameters D₆ᵢ; in such a way, the curves e and f form the second pair of biological impedance spectrum curve.

S8, analyzing the three pairs of biological impedance spectrum curves by means of weighting method and determining whether the probe is located at the juncture of different biological tissues.

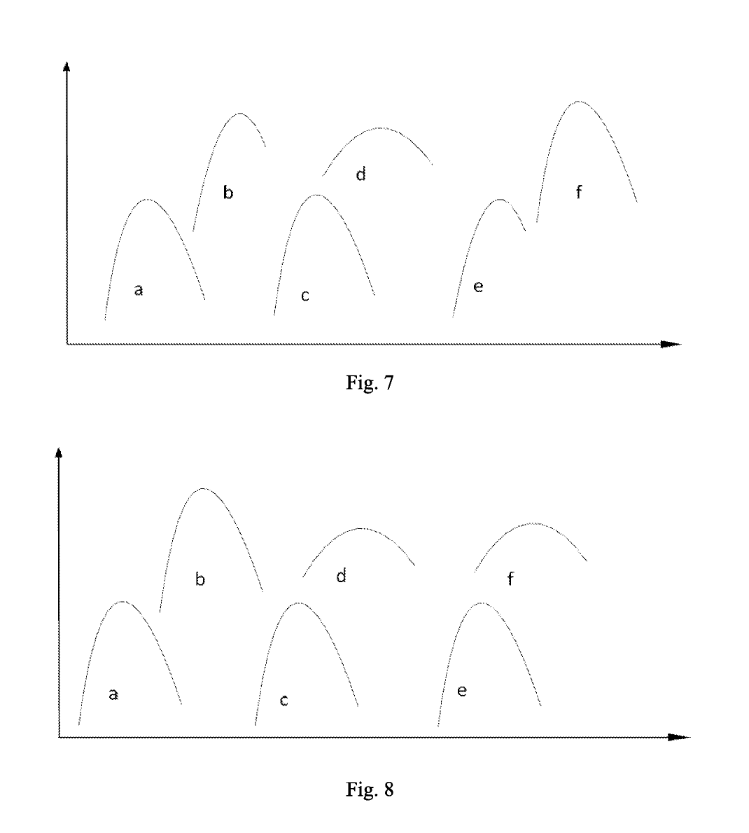

As shown in FIG. 2, if the contacting between the probe and the different biological tissues is under Q1 state, three pairs of biological impedance spectrum curves are obtained according to the above-mentioned method, as shown in FIG. 7. Specifically, shapes and trends of the curve a and curve b are different from one another, shapes and trends of the curve c and curve d are different from one another, and shapes and trends of the curve e and curve f are different from one another, by this token, the juncture of different biological tissues is located at or close to the center of the probe.

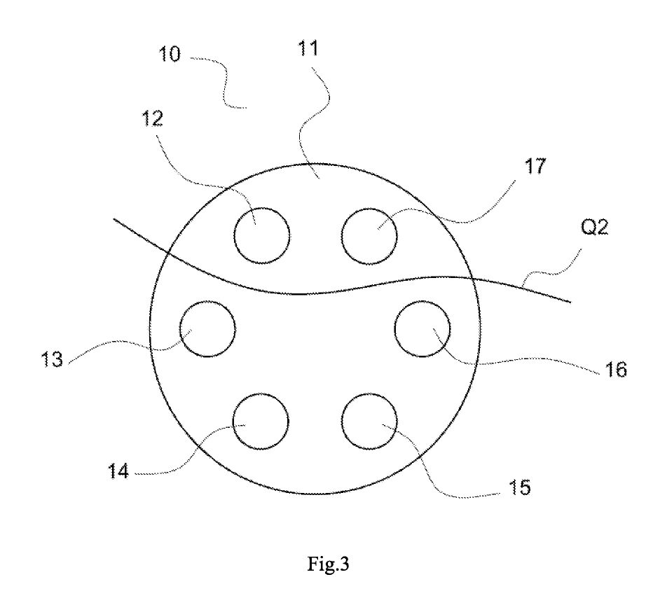

As shown in FIG. 3, if the contacting between the probe and the different biological tissues is under Q2 state, three pairs of biological impedance spectrum curves are obtained according to the above-mentioned method, as shown in FIG. 8. Specifically, shapes and trends of the curve a and curve b are the same, while shapes and trends of the curve c and curve d are different from one another, shapes and trends of the curve e and curve f are different from one another, by this token, the juncture of different biological tissues is far from the center or edge of the probe.

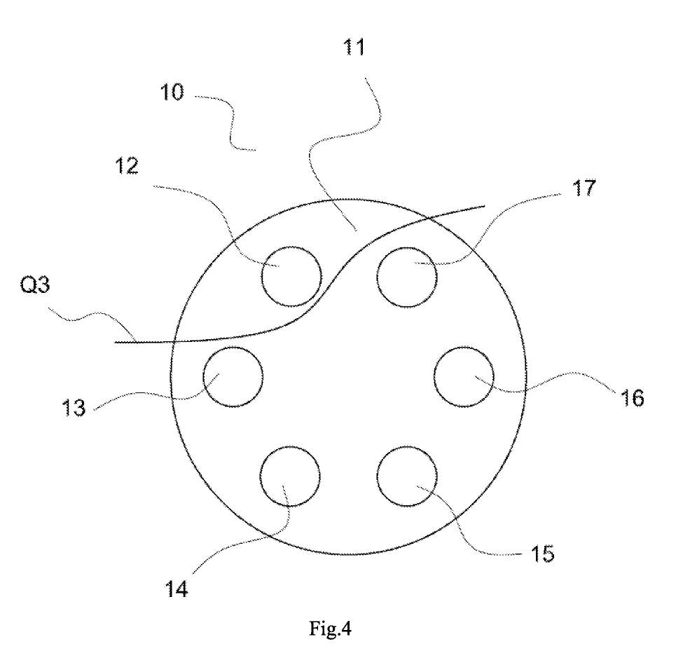

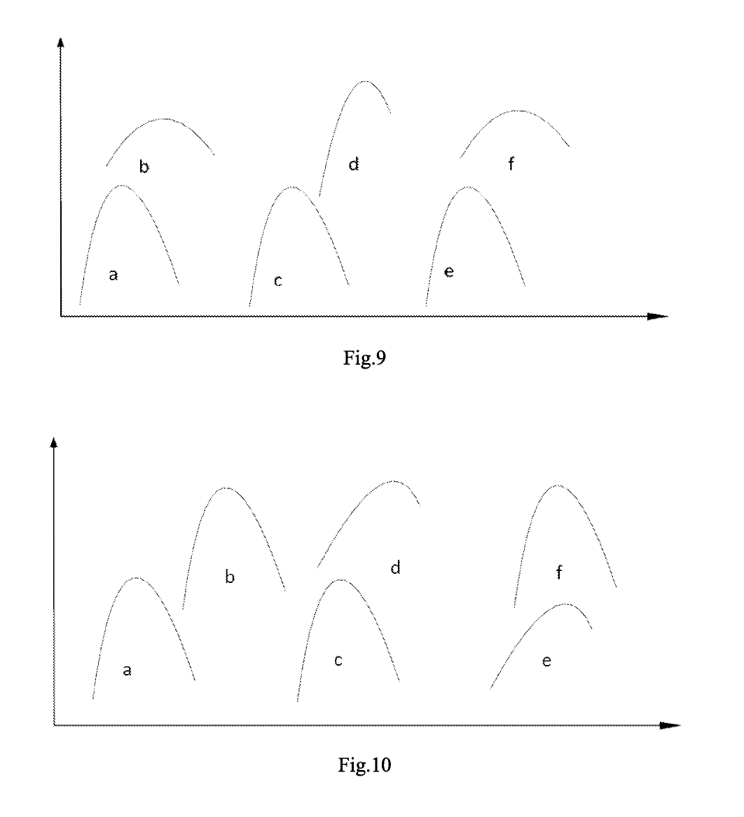

As shown in FIG. 4, if the contacting between the probe and the different biological tissues is under Q3 state, three pairs of biological impedance spectrum curves are obtained according to the above-mentioned method, as shown in FIG. 9. Specifically, shapes and trends of the curve a and curve b are different from one another, shapes and trends of the curve c and curve d are different from one another, shapes and trends of the curve e and curve f are different from one another, but the pair of curves c and d have the same shapes and trends with the pair of curves e and f. By this token, the juncture of different biological tissues is located at or close to the edge of the probe.

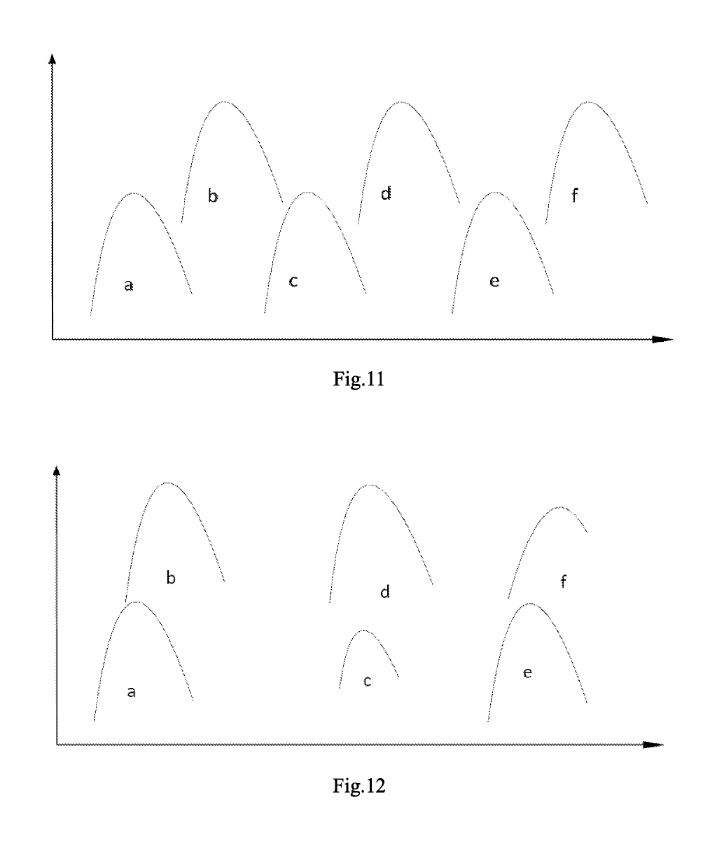

As shown in FIG. 11, if the probe is disposed on the congeneric biological tissues, the shapes and trends of the biological impedance spectrum curves a, b, c, d, e and f should be the same.

FIGS. 7-9 just show the ideal states, but some differences may appear in the actual case, nevertheless, the shapes and the trends of the actual curves are substantially consistent. Thus the present invention may use weighting method to analyze the three pairs of biological impedance spectrum curves. The weight method follows. Of course, other analyzing methods also can be used.

If at least two pairs of biological impedance spectrum curves in the three pairs are different, namely the weights is no less than 50%, it's determined that the probe is located the juncture of different biological tissues, such as human body tissues or animal body tissues. For example, the shapes and the trends of the curves a and b are substantially the same, while the shapes and the trends of the curves c and b are different, the shapes and the trends of the curves e and d are different; or the shapes and the trends of the curves a and b, c and b, e and e are different individually.

If at least two pairs of biological impedance spectrum curves in the three pairs are the same, namely the weights is less than 50%, it's determined that the probe is located the juncture of the congeneric biological tissues, such as human body tissues or animal body tissues. For example, the shapes and the trends of the curves a and b are different, while the shapes and the trends of the curves c and b are substantially the same, the shapes and the trends of the curves e and d are substantially the same; or the shapes and the trends of the curves a and b, c and b, e and e are substantially the same, individually.

By this token, FIGS. 7, 8, 9 and 10 show that the probe is placed on junctures of different biological tissues (human body tissues or animal body tissues), while FIGS. 11 and 12 shows that the probe is placed on junctures of the congeneric biological tissues ((human body tissues or animal body tissues). The present invention may improve the accuracy of the collection signals, effectively reduce the influence of the contact impedance between the probe and the measured tissues, and improve the accuracy of judging whether the probe is placed on junctures of different biological tissues.

In other embodiments of the measurement method, the above steps S3˜S6 may be replaced by the following steps.

S3, selecting the excitation 30 by the multiple-selection switch system 20, and applying the excitation 30 between the first electrode 12 and the fourth electrode 15;

collecting a first electrical parameter D₁ᵢ between the second electrode 13 and the third electrode 14, and a second electrode parameter D₂ᵢ between the fifth electrode 16 and the sixth electrode 17, by the signal collection circuit, with the excitation is f₁, f ₂, . . . f_{N} respectively;

storing and recording the group of the electrical parameters, namely N first electrical parameters and N second electrical parameters.

S4, selecting the excitation 30 by the multiple-selection switch system 20, and applying the excitation 30 between the second electrode 13 and the fifth electrode 16;

collecting a third electrical parameter D₃ᵢ between the third electrode 14 and the fourth electrode 15, and a fourth electrode parameter D₄ᵢ between the first electrode 12 and the sixth electrode 17, by the signal collection circuit, with the excitation is f₁, f₂, . . . f_{N} respectively; and

storing and recording the group of the electrical parameters, namely N third electrical parameters D₃ᵢ and N fourth electrical parameters D₄ᵢ.

S5, selecting the excitation 30 by the multiple-selection switch system 20, and applying the excitation 30 between the third electrode 14 and the sixth electrode 17;

collecting a fifth electrical parameter D₅ᵢ between the fourth electrode 15 and the fifth electrode 16, and a sixth electrode parameter D6i between the first electrode 12 and the second electrode 13, by the signal collection circuit, with the excitation is f₁, f₂, . . . f_{N} respectively; and

storing and recording the group of the electrical parameters, namely N fifth electrical parameters D₅ᵢ and N fourth electrical parameters D₆ᵢ.

The above descriptions are considered to be the preferred embodiments, it is to be understood that the invention is not to be limited to the disclosed embodiments, but on the contrary, is intended to cover various modifications and equivalent arrangements included within the spirit and scope of the invention.

1. A biological impedance measurement probe based on spectral characteristic, comprising a substrate and at least six electrodes embedded in the substrate, wherein the six electrodes includes a first electrode, a second electrode, a third electrode, a fourth electrode, a fifth electrode and a sixth electrode, the first electrode is arranged opposite to the fourth electrode, the second electrode is arranged opposite to the fifth electrode, and the third electrode is arrange opposite to the sixth electrode.

2. The biological impedance measurement probe according to claim 1, wherein the six electrodes are circumferentially distributed on the substrate.

3. The biological impedance measurement probe according to claim 2, wherein surfaces of the six electrodes are flush with a surface of the substrate, and spaces between two adjacent electrodes are identical.

4. The biological impedance measurement probe according to claim 2, wherein a central angle between two electrodes that are opposite to each other is 180°.

5. The biological impedance measurement probe according to claim 1, wherein an excitation is applied between the electrodes that are opposite to each other.

6. A biological impedance measurement system based on spectral characteristic, comprising:
a probe, comprising a substrate and at least six electrodes embedded in the substrate, the six electrodes including a first electrode, a second electrode, a third electrode, a fourth electrode, a fifth electrode and a sixth electrode;
an excitation source, adapted for applying N excitations with different frequencies to the first electrode and the fourth electrode that are opposite to each other, the second electrode and the fifth electrode that are opposite to each other, and the third electrode and the sixth electrode that are opposite to each other, and the frequencies being expressed by f, i=1, 2, 3 . . . N;
a signal collection circuit, adapted for measuring a first electrical parameter D₁ᵢ between the second electrode and the third electrode, a second electrical parameter D₂ᵢ between the fifth electrode and the sixth electrode, a third electrical parameter D₃ᵢ between the third electrode and the fourth electrode, a fourth electrical parameter D₄, between the first electrode and the sixth electrode, a fifth electrical parameter D₅ᵢ between the fourth electrode and the fifth electrode, and a sixth electrical parameter D₆ᵢ between the first electrode and the second electrode;
a multiple-selection switch system, adapted for controlling the connection and disconnection among the six electrodes, the excitation source and the signal collection circuit; and
a spectrum analyzer, adapted for recording and storing three pairs of the electrical parameters D₁ᵢ and D₂ᵢ, D₃ᵢ and D₄ᵢ, D₅ᵢ and D₆ᵢ, and performing logic judgment and statistical analysis to the three pairs of the electrical parameters.

7. The measurement system according to claim 6, wherein the six electrodes are circumferentially distributed on the substrate.

8. The measurement system according to claim 7, wherein surfaces of the six electrodes are flush with a surface of the substrate, and spaces between two adjacent electrodes are identical.

9. The measurement system according to claim 7, wherein a central angle between two electrodes that are opposite to each other is 180°.

10. A measurement method by means of the biological impedance measurement probe based on spectral characteristic according to claim 1, comprises:
step 1, disposing a probe on biological tissues, with the probe contacting with the biological tissues;
step 2, selecting N frequencies in a testing frequency range of fₘ˜fₙ, and selecting an excitation source with a frequency fᵢ ∈[fₘ, fₙ], and=1, 2, 3 . . . N, fₘ<fₙ;
step 3, controlling and respectively applying excitations with N frequencies to the first electrode and the fourth electrode, the second electrode and the fifth electrode, and the third electrode and the sixth electrode, by means of the multiple-selection switch system; collecting a first electrical parameter D₁ᵢ, between the second and the third electrodes and a second electrical parameter D₂ᵢ between the fifth and the sixth electrodes when the excitation is applied to the first electrode and the fourth electrode, a third electrical parameter D₃ᵢbetween the third and the fourth electrodes and a fourth electrical parameter D₄ᵢ between the first and the sixth electrodes when the excitation is applied to the second and the fifth electrodes, and a fifth electrical parameter D₅ᵢ between the fourth and the fifth electrodes and a sixth electrical parameter D₆ᵢbetween the first and the second electrodes when the excitation is applied to the third and the sixth electrodes, by the signal collection circuit;
step 4, performing curve fitting to N first electrical parameters D₁ᵢ and N second electrical parameters D₂ᵢ, N third electrical parameters D₃ᵢ and N fourth electrical parameters D₄ᵢ, N fifth electrical parameters D₅ᵢ and N sixth electrical parameters D₆ᵢ to obtain three pairs of biological impedance spectrum curves; and
step 5, analyzing the three pairs of biological impedance spectrum curves by means of weighting methods to determine whether the biological tissues are different or not.

11. The measurement method according to claim 10, wherein the step 3 further comprises:
step 31, when i=1, fᵢ=fᵢ, applying an excitation with a frequency fᵢ between the first electrode and the fourth electrode, between the second electrode and the fifth electrode, and between the third electrode and the sixth electrode, by the multiple-selection switch system;
step 32, collecting a first electrical parameter D₁ᵢ between the second electrode and the third electrode, a second electrode parameter D₂ᵢ between the fifth electrode and the sixth electrode , a third electrode parameter D₃ᵢ between the third electrode 14 and the fifth electrode 15, a fourth electrode parameter D₄ᵢ between the first electrode 12 and the sixth electrode 17, a fifth electrode parameter D₅ᵢ between the fourth electrode 15 and the fifth electrode 16, and a sixth electrode parameter D₆ᵢ between the first electrode 12 and the second electrode 13, by the signal collection circuit;
step 33, storing and recording three groups of the electrical parameters D₁ᵢ and D₂ᵢ, D₃ᵢ and D₄ᵢ, D₅ᵢ and D₆ᵢ;
step 34, when i≦N, i=i+1, repeating the step S31 to step S33.

12. The measurement method according to claim 10, further comprising obtaining N first electrical parameters D₁ᵢ between the second and the third electrodes and performing statistical analysis to the N first electrical parameters D₁ᵢ to obtain a curve a, and obtaining N second electrical parameters D₂ᵢ between the fifth and the sixth electrodes and performing statistical analysis to the N second electrical parameters D₂ᵢ to obtain a curve b, when excitations with N different frequencies f ᵢ are applied to the first and the fourth electrodes, wherein the curves a and b constitute a first pair of biological impedance spectrum curve.

13. The measurement method according to claim 10, further comprising obtaining N third electrical parameters D₃ᵢ between the third and the fourth electrodes and performing statistical analysis to the N third electrical parameters D₃ᵢ to obtain a curve c, and obtaining N fourth electrical parameters D₄ᵢ between the first and the sixth electrodes and performing statistical analysis to the N fourth electrical parameters D₄ᵢ to obtain a curve d, when excitations with N different frequencies f ᵢ are applied to the second and the fifth electrodes, wherein the curves c and d constitute a second pair of biological impedance spectrum curve.

14. The measurement method according to claim 10, further comprising obtaining N fifth electrical parameters D₅ᵢ between the fourth and the fifth electrodes and performing statistical analysis to the N fifth electrical parameters D₅ᵢ to obtain a curve e, and obtaining N sixth electrical parameters D₆ᵢ between the first and the second electrodes and performing statistical analysis to the N sixth electrical parameters D₆ᵢ to obtain a curve f, when excitations with N different frequencies f ᵢ are applied to the third and the sixth electrodes, wherein the curves e and f constitute a third pair of biological impedance spectrum curve.
